(19) United States
(12) Plant Patent Application Publication
(10) Pub. No.: US 2009/0007304 P1
(43) Pub. Date: Jan. 1, 2009

(54) Hybrid tea rose plant named 'JACeleco'

(75) Inventor: Keith W. Zary, Thousand Oaks, CA (US)

Correspondence Address: KLARQUIST SPARKMAN, LLP, 121 SW SALMON STREET, SUITE 1600, PORTLAND, OR, 97204, US

(73) Assignee: Jackson & Perkins Wholesale, Inc.

(21) Appl. No.: 11/824,549

(22) Filed: Jun. 29, 2007

(51) Int. Cl. A01H 5/00

(52) U.S. Cl. PLT/131

(57) ABSTRACT

Hybrid tea rose plant having flowers with large flower size; compact, well-branched plant with glossy, dark green foliage; resistant to rust and powdery mildew and ability to own root from softwood cuttings.

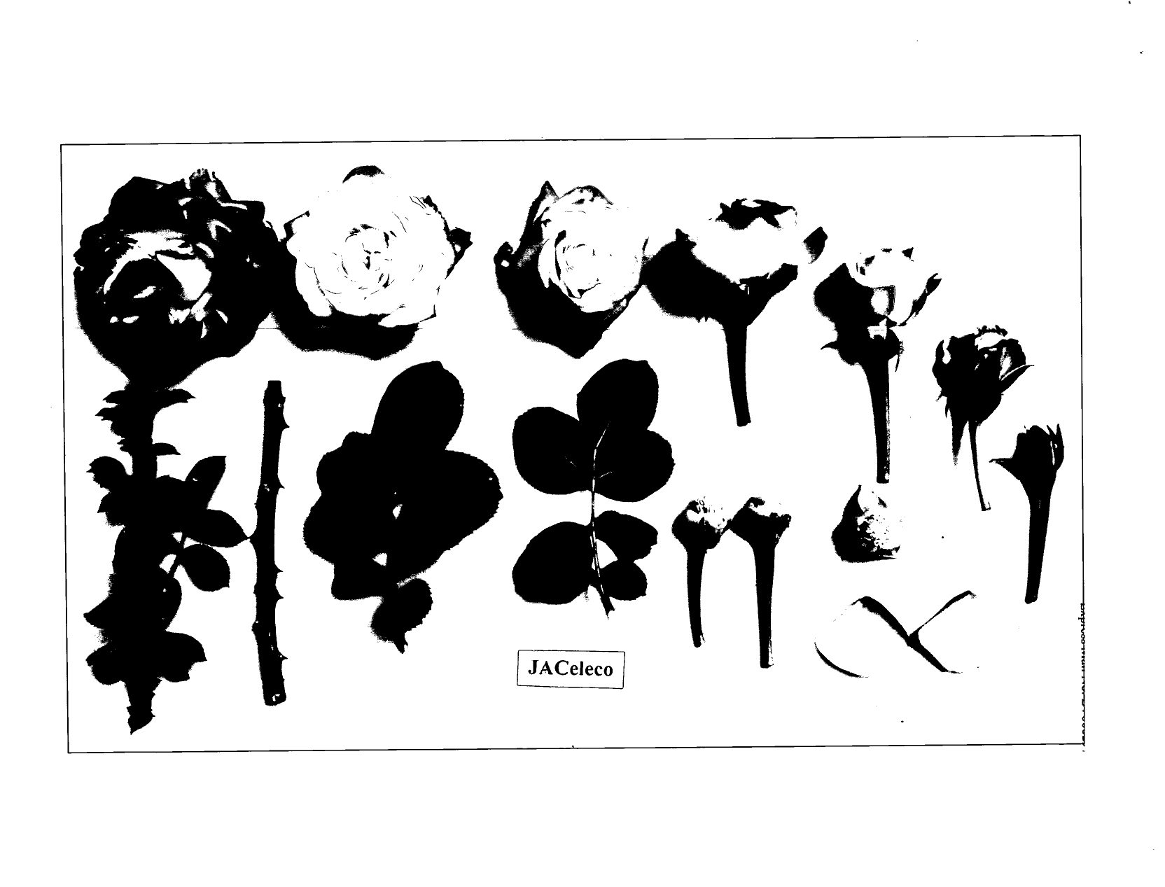

### GENUS AND SPECIES

Rosa hybrida

### VARIETY DENOMINATION

‘JACeleco’

### ORIGIN OF THE INVENTION

The present invention relates to a new and distinct variety of a hybrid tea rose plant named ‘JACeleco’ which was originated by me by crossing the variety ‘Twobe’ seed parent, (PP 7901), with the variety ‘JACmag’ pollen parent (PP 3998).

### BACKGROUND OF THE INVENTION

The primary objective of this breeding was to produce a new hybrid tea rose variety having the compact well-branched habit of the female (seed) parent combined with the large flower size of the male (pollen) parent.

### BRIEF SUMMARY OF THE INVENTION

The objective was substantially achieved, along with other desirable improvements, as evidenced by the following unique combination of characteristics that are outstanding in the new variety and that distinguish it from its parents, as well as from all other varieties of which I am aware:
1. Large flower size.
2. Compact, well-branched plant.
3. Glossy, dark green foliage.
4. Resistance to powdery mildew and rust.
5. Ability to own root from softwood cuttings.

Asexual reproduction of this new variety by cuttings, as performed at Wasco, Calif., shows that the foregoing characteristics and distinctions come true to form and are established and transmitted through succeeding propagations.

### COMPARISON WITH PARENTS

‘Twobe’ differs in plant habit. ‘JACeleco’ is shorter and rounded in habit. ‘JACeleco’ also has larger flower. ‘JACmag’ differs in flower petal count. ‘JACeleco’ has 30 petals under normal growing conditions compared to 25 petals for ‘JACmag’

### COMPARISON WITH SIMILAR VARIETY

The closest variety is believed to be ‘Twobe’ which is compared above.

### BRIEF DESCRIPTION OF THE ILLUSTRATION

The accompanying illustration shows typical specimens of the vegetative growth and flowers of this new variety in different stages of development, depicted in color as nearly true as it is reasonably possible to make the same in a color illustration of this character.

### DETAILED BOTANICAL DESCRIPTION

The following is a detailed description of my new rose cultivar with color descriptions using terminology in accordance with The Royal Horticultural Society (London) Colour Chart© 2001, except where ordinary dictionary significance of color is indicated.
Parentage:
   Seed parent.—‘Twobe’ (PP 7901).
   Pollen parent.—‘JACmag’ (PP 3998).

### FLOWER

Observations made from January 2006 to June 2007 from plants commencing at two to three years of age grown in a garden environment at Somis, Calif.
Blooming habit: Continuous.
Bud:
   Size.—About 45 mm long and 30 mm in diameter when the petals start to unfurl.
   Form.—The bud form is long, pointed ovoid.
   Color.—When sepals first divide, bud color is Red Group 43B with patches of Red Group 43C and Red Group 43D in areas not as exposed to sun. When half blown, the upper sides of the petals are Red Group 43B on the petal edges with Red Group 49B to Red Group 49C on the surfaces just exposed to the sun, and the lower sides of the petals are Red Group 43B on the edges and Red Group 49C over the rest of the petal.
   Sepals.—Color: Upper surface: Green Group 138B that in bright sun can be overlain with Greyed-Purple Group 184B. Lower surface: Yellow-Green Group 146C Length: 25 mm. Width: 13 mm. Shape: Subulate. Surface texture: Hoary. There are three lightly appendaged sepals. There are two unappendaged sepals with hairy edges.
   Receptacle.—Color: Yellow-Green Group 146B which may be overlain with Greyed-Purple Group 184B. Shape: Funnel. Size: Large, about 17 mm long×12 mm in diameter. Surface: Smooth.
   Peduncle.—Length: Short, averaging about 45 mm. Surface: Smooth. Color: Yellow-Green Group 146B. Strength: Stiff, erect, heavy.
Bloom:
   Size.—Large. Average open diameter is about 135-140 mm.
   Borne.—Singly.
   Stems.—Strength: Strong. Average length is about 35-45 cm with a diameter of 7-9 mm.
   Form.—When first open: High centered. Permanence: Retains its form to the end, but outer petals curl slightly.
   Petalage.—Number of petals under normal conditions: 30.
   Color.—The upper sides of the petals are Red Group 43B on the petal edges with Red Group 49B to Red Group 49C on the surfaces just exposed to sunlight. The reverse sides of the petals are Red Group 43B on the edges and Red Group 49C over the rest of the petals. The base of the petals has an approximately 5 mm×5 mm Yellow Group 8C color spot. The major color on the upper sides is Red Group 43B.
   Variegations.—None observed.
   Discoloration.—The general tonality at the end of the first day: Little change. At the end of the third day: A gradual intensification of color as sunlight hits the more exposed petal. The Red Group 43B spreads across the petal surface. Further aging: Finishes mostly Red Group 43B.
   Fragrance.—Slight, spicy.
Petals:
   Texture.—Thick.
   Surface.—Smooth.
   Shape.—Round.
   Margin.—Entire.
   Apex shape.—Obtuse.
   Base shape.—Rounded.
   Form.—Tips slightly recurved, edges slightly quilled.
   Arrangement.—Imbricated.
   Petaloids.—Number: 3-5. Size: 5-9 mm long×3-5 mm wide. Color: Red Group 49C.
   Persistence.—Petals drop off cleanly before drying.
   Lastingness.—On the plant: Long (about 7 days). As a cut flower: Long (about 9 days).
Reproductive parts:
   Stamens.—Number: 140-160. Length: 9-11 mm. Anthers: Length: 2-3 mm. Color: Yellow Group 12C. Arrangement: Regular around styles. Filaments: Color: Red-Purple Group 60D. Length: 7-9 mm Pollen: Color: Greyed-Orange Group N170B.
   Pistils.—Number: 80-90. Length: 6-7 mm. Styles: Color: Greyed-White Group 157C. Length: 5-6 mm. Stigmas: Color: Yellow-White Group 158C. Length: 1 mm.
   Hips.—None observed.

### PLANT

Form: Bush.
Growth: Vigorous, branching, compact.
Age to maturity: Two years.
Mature plant:
   Height.—5 ft.
   Width.—3-½ ft.
Rootstock: When growing on ‘Dr. Huey’ rootstock
Foliage: Number of leaflets on normal mid-stem leaves: 5-7.
   Size.—Large. About 15-17 cm long×12-14 cm wide.
   Quantity.—Normal.
   Color.—New foliage: Upper side: Greyed-Purple Group 183A. Lower side: Blend of Yellow-Green Group 144B and Greyed-Red Group 181A. Old foliage: Upper side: Green Group 135A. Lower side: Yellow-Green Group 144A.
Leaflets:
   Size.—About 6-8 cm long×5-7 cm wide.
   Shape.—Round, broadly oval.
   Base shape.—Rounded.
   Apex shape.—Cuspidate.
   Texture.—Leathery, glossy.
   Edge.—Serrated.
   Serrations.—Single, small.
   Petiole.—Color: Yellow-Green Group 144B.
   Petiole rachis.—Color: Yellow-Green Group 144B. Underside: Prickles, very few and small.
   Auricles.—Subulate.
   Stipules.—Length: 3-4 cm., glandular.
   Disease resistance.—Resistant to mildew and rust, susceptible to blackspot under normal growing conditions.
   Pest resistance.—Not observed.
   Vein color.—Green Group 135A.
   Venation pattern.—Reticulate.
Wood:
   New wood.—Color: Yellow-Green Group 144A with occasional streak of Greyed-Red Group 181A. Bark: Smooth.
   Old wood.—Color: Yellow-Green Group 144A. Bark: Smooth.
Prickles:
   Quantity.—Ordinary on main canes from base: 25-30 per 150 mm of cane. Ordinary on laterals from main canes.
   Form.—Straight.
   Color when young.—Greyed-Red Group 182B.
   Color when mature.—Greyed-Orange Group 166D.
Small prickles:
   Quantity.—On main stalks: Few. On laterals: Few.
Winter hardiness: Hardy to Zone 5.

I claim:

1. A new and distinct variety of hybrid tea rose plant, substantially as herein shown and described, characterized particularly by its large flower size; compact, well-branched plant with glossy, dark green foliage; resistant to rust and powdery mildew and ability to own root from softwood cuttings.
